SEQUENCE LISTING (8 sequences)

SEQ ID NO: 1 - protein, 166 aa, Homo sapiens
CDLPQTHSLGSRRTLMLLAQMRRISLFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMRSFSLSTNLQESLRSKEG

SEQ ID NO: 2 - protein, 166 aa, Homo sapiens
SYNLLGFLQRSSNFQSQKLLWQLNGRLEYCLKDRMNFDIPEEIKQLQQFQKEDAALTIYEMLQNIFAIFRQDSSSTGWNETIVENLLANVYHQINHLKTVLEEKLEKEDFTRGKLMSSLHLKRYYGRILHYLKAKEYSHCAWTIVRVEILRNFYFINRLTGYLRNG

SEQ ID NO: 3 - DNA, 31 bp, Artificial Sequence
  oligonucleotide
ggtggtcatatgtgtgatctgcctcaaaccc

SEQ ID NO: 4 - DNA, 46 bp, Artificial Sequence
  oligonucleotide
ggtggttgctcttccgcacccttccttacttcttaaactttcttgc

SEQ ID NO: 5 - protein, 166 aa, Artificial Sequence
  IFNalpha2b hydrazide
CDLPQTHSLGSRRTLMLLAQMRRISLFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMRSFSLSTNLQESLRSKEX

SEQ ID NO: 6 - protein, 13 aa, Artificial Sequence
  positive control peptide sequence  for ES MS analysis after NEM incubation
CERGDKGYVPSVF

SEQ ID NO: 7 - DNA, 9 bp, Artificial Sequence
  5′-terminal nucleotide sequence containing NdeI restriction endonuclease recognition site
ggtggtcat

SEQ ID NO: 8 - DNA, 18 bp, Artificial Sequence
  3′-terminal nucelotide sequence containing SapI restriction endonuclease recognition site
tgcggaagagcaaccacc

(19) United States
(12) Patent Application Publication
(10) Pub. No.: US 2012/0128629 A1
(43) Pub. Date: May 24, 2012

(54) METHOD OF LABELLING INTERFERONS WITH PEG

(75) Inventors: Jennifer Roberts, East Lothian (GB); Graham Cotton, East Lothian (GB)

(73) Assignee: Almac Sciences (Scotland) Limited, Edinburgh (GB)

(21) Appl. No.: 13/265,032

(22) PCT Filed: May 17, 2010
(86) PCT No.: PCT/GB2010/000981

§ 371 Date: Nov. 15, 2011

(30) Foreign Application Priority Data
May 15, 2009 (GB) 0908393.2

(51) Int. Cl. A61K 38/21; A61P 35/00; A61P 1/16; A61P 37/02; C07K 14/555; C07K 14/565

(52) U.S. Cl. 424/85.4; 530/351

(57) ABSTRACT

A method of site specific labelling of an interferon molecule is provided. The method comprises the steps: a) providing a label molecule comprising a PEG moiety having an aldehyde or ketone moiety; b) providing an interferon molecule having a C terminal hydrazide moiety; and c) allowing the aldehyde or ketone moiety of the PEG moiety to react with the C terminal hydrazide of the interferon molecule to form a labelled interferon molecule, which comprises a PEG moiety attached to the C terminus of the interferon molecule via a hydrazone bond. Interferon molecules labelled using such a method are also described.

### FIELD OF THE INVENTION

This application relates to a method of site specific modification of peptides, proteins etc. In particular it relates to a method of labelling proteins such as interferons with PEG.

### BACKGROUND TO THE INVENTION

Recombinant protein therapeutics have emerged as an effective treatment for a variety of conditions ranging from cancer to metabolic disorders and autoimmune diseases, but they are commonly limited by their pharmacokinetics and immunogenicity. Consequently a number of strategies have been developed to overcome these, including glycosylation, attachment of albumin, cyclisation and PEGylation. Protein glycosylation is the attachment of carbohydrate(s), which can aid protein stability and give some protection from proteolysis and immune recognition (Doores et al., 2006). Human albumin is the most prevalent serum protein in the circulation and has an unusually long half life of ˜19 days. Consequently genetic fusion of albumin to the N or C terminus of a protein is well tolerated and the resulting fusion protein has a significantly increased half life (Subramanian et al., 2007).

PEGylation, the covalent attachment of poly ethylene glycol (Veronese and Mero, 2008) is arguably the most widely used and accepted method for improving the pharmacokinetics of proteins. PEG is a polymer based on the repeating unit (—C₂H₃—O—)ₙ available in a wide range of molecular weights with low polydispersity and can be both linear and branched. Its high flexibility and hydration means it has a large hydrodynamic radius and significantly increases the size of the protein to which it is attached, in turn significantly decreasing its renal clearance. In addition potentially immunogenic epitopes and protease cleavage sites are masked reducing immunogenicity and proteolysis respectively. Further, PEGylation can improve protein solubility and stability substantially.

However, the known methods for PEGylation are generally non-site selective, using electrophillic PEG derivatives that undergo acylation or alkylation with protein nucleophiles, for example amino groups on lysine side chains (Roberts et al., 2002). This often generates a heterogeneous protein preparation, where 1 PEG molecule is attached to the protein at a number of different sites to generate different positional isomers. At some positions within the protein attachment of PEG prevents or compromises receptor binding. In addition multi-PEGylated species may also be present, whereby a number of PEG molecules are attached to the same protein at different sites. Consequently, the total activity of the PEGylated protein preparation is reduced compared to the unmodified protein. As a result, there is a need for methods for site specific protein PEGylation; by incorporating a single PEG moiety at a defined position within the protein sequence, the deleterious effects associated with non-selective PEGylation (ie preparations containing multiple PEG positional isomers) may be overcome.

The most common method employed to introduce a label into a protein in a site-specific fashion is to engineer a unique free cysteine into the primary sequence at the position for modification. The sulphydryl side-chain of this free cysteine is then reacted with maleimide derivatives of the label to afford site-specific modification. However, this requires all other naturally occurring free cysteines within the primary sequence to be removed through amino acid mutagenesis. In addition if the protein naturally contains disulphide bonds, the addition of an extra cysteine within the molecule may interfere with the correct folding of the protein.

As a consequence, other approaches have been investigated for the site-specific PEGylation of proteins. Marsac et al., 2006 describe the attachment of PEG to the N terminus of a protein by native chemical ligation between a PEG-thioester and the N terminal cysteine on target protein. This requires addition of an N terminal cysteine onto the protein sequence if not already present, which may interfere with protein folding in cysteine containing proteins. Kinstler et al., 2002 describe attachment of PEG to the N terminus by reductive alkylation of protein with PEG aldehyde under acidic conditions under which only the α-amino N terminal amine is reactive c.f. pka lower than c-amino group of lysine resides (Kinstler et al., 2002). However, there is still potential to get some ε-amino PEGylation with this method. Incorporation of PEG into disulfide bridges is suggested in Brocchini et al., 2008. This involves initial reduction of the disulfide to release the 2 cysteine thiols, followed by bis-alkylation to give a 3 carbon bridge to which PEG is covalently attached. However, this approach is limited to proteins containing solvent exposed disulfide bonds. A further method is PEGylation at a natural glycosylation site, in which the recombinant protein, expressed in E. coli, is glycosylated enzymatically and PEG is attached via the glycan at the natural glycosylation site (DeFrees et al., 2006). This approach is limited to proteins that are naturally glycosylated. Zhang et al., 2009, describe protein C terminal PEGylation through thioacid/azide amidation; here the protein is expressed as a VMA intein CBD fusion protein and hydrothiolitically cleaved with Na₂S to give the thioacid, which can then be reacted with PEG-sulfonazide. However, the thioacid protein is prone to hydrolysis during the labelling reaction, yielding the unlabelled C-terminal carboxylic acid derivative of the protein as a by product.

Xie and Schultz, 2006 describe the incorporation of unnatural amino acids with reactive chemical groups during protein expression in E. coli, that can enable subsequent attachment of PEG functionalities. This is achieved using a unique codon (e.g. the amber nonsense codon, UAG) and the corresponding transfer RNA:aminoacyl-tRNA-synthetase pair engineered into E. coli.

WO 2005/110455 and WO2004/076474 describe the use of PEGylated interferon for the treatment of viral infections. Likewise, US2005/059129 describes PEGylated interferons. However, the methods described for the PEGylation of interferon by these documents are not site specific.

There are currently nine PEGylated protein therapeutics approved for therapeutic use (Veronese & Mero 2008 Biodrugs 22(5) p315) including PEGylated versions of Adenosine deaminase, G-CSF, Erythropoietin, IFNalpha 2a and IFNalpha 2b.

There are two approved PEGylated versions of IFNalpha2 used in the treatment of hepatitis C virus and under clinical evaluation for use in certain cancers (Ferrantini et al., 2007). Pegasys® (Hoffmann La Roche) is recombinant IFNalpha2a attached to a branched 40 kDa PEG-NHS. It comprises 9 positional PEG isomers. However, it retains only 7% activity of the unPEGylated IFNalpha2a in in vitro assays (Dhalluin et al., 2005; Foser et al., 2003). Peglntron® (Schering Plough) is recombinant IFNalpha2b attached to a single chain 12 kDa succinimidyl carbonate PEG resulting in 95% monoPEGylated protein comprising 14 positional isomers of which histidine 34 makes up 47.8%. Peglntron® retains 28% anti-viral activity in in vitro assays compared to unPEGylated IFNalpha2b (Wang et al., 2002).

Betaseron® (Betaferon in EU) (Bayer Shering Pharma) and more recently Extavia (Novartis) are recombinant IFNbeta1b proteins used in the treatment of multiple sclerosis and are under clinical evaluation for treatment of other diseases including hepatitis and certain cancers (Fine et al., 1997; Fukutomi et al., 2001). However, a particular problem with IFNbeta1b is that it is rapidly cleared from the blood necessitating frequent administration regimes that can result in injection site necrosis and decreased patient compliance. Neutralizing antibodies are also a problem with 45% of patients developing these in one 2 year study. There is currently no PEGylated IFNbeta1b version approved. However, work in this area to date includes random PEGylation of primary amines that results in a mixture of 5 positional isomers with 24-31% activity of unmodified IFNbeta1b, and PEGylation of (predominantly) the N terminal amine (35% activity). Attempts at site specific PEGylation using engineered free Cys residues at either position 79 (the natural glycosylation site) or the N or C termini were unsuccessful due to insufficient site specific attachment (Basu et al., 2006). Another strategy used bicin (bis-N-2-hydroxyethylglycinamide) linkers to randomly attach 2-3 PEG molecules. Under physiological conditions rapid hydrolysis of the bicin releases the PEG to leave the active IFNbeta1b. However, the activity of these releasable PEGylated forms was only between 7 and 27% that of unmodified IFNbeta1b (Zhao et al., 2006).

In order to improve the efficacy and stability of protein thereapeutics, there is a clear need for means to prolong the effectiveness of interferon therapeutics, for example by delaying renal clearance, decreasing immunogenicity, and/or decreasing proteolysis. However, although there are a number of methods known in the art, each has its disadvantages, for example in the requirement of introduction of additional chemical moieties, imitations in the site of modification, effects on protein folding and effects on activity.

### SUMMARY OF THE INVENTION

The present inventors have investigated alternative methods of stabilising interferons for use therapeutically. They have developed a novel method of PEGylating interferons which is site specific and which, for the interferons tested, as described in the Examples resulted in PEGylated interferons with antiviral activity considerably greater than corresponding interferons PEGylated using conventional techniques, and indeed approaching that of the non-PEGylated interferons. This was facilitated by generating novel oxocarboxylic acid, for example pyruvoyl, derivatives of the PEG functionality. C terminal hydrazide recombinant proteins, were reacted with pyruvoyl PEG to generate the site-specifically C terminal PEGylated protein in good yield, to which the PEG functionality was directly attached to the C terminus of the protein through a hydrazone bond. The hydrazone bond can be further stabilised by reduction, for example under mild conditions with sodium cyanoborohydride.

Accordingly, in a first aspect of the present invention, there is provided a method of site specific labelling of an interferon molecule, wherein said method comprises the steps:

a) providing a label molecule, the label molecule comprising a PEG moiety having an aldehyde or ketone moiety;
b) providing an interferon molecule, the interferon molecule having a C terminal hydrazide moiety;
c) allowing the aldehyde or ketone moiety of the PEG moiety to react with the C terminal hydrazide of the interferon molecule to form a labelled interferon molecule, which comprises a PEG moiety attached to the C terminus of the interferon molecule via a hydrazone bond.

In one embodiment of the invention, the hydrazone bond has Formula I:

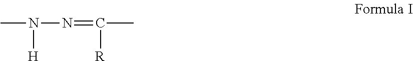

where R is H or any substituted or unsubstituted, preferably unsubstituted, alkyl group.

In one embodiment, the method comprises:

d) reacting the labelled interferon molecule produced in step (c) with a reducing agent, wherein the hydrazone bond is reduced to the corresponding substituted hydrazine.

The reduction of the hydrazone bond to its reduced form is shown schematically below:

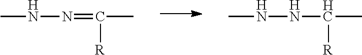

Any suitable reducing agent may be used in step (d). In one embodiment, in which the hydrazone bond is reduced, the reducing agent is cyanoborohydride.

In the method of the invention, a PEG molecule having any suitable aldehyde or ketone moiety may be used. In one embodiment, the aldehyde or ketone moiety is an α-diketone or an α-keto-aldehyde group.

In another embodiment of the invention, the PEG moiety having an aldehyde or ketone moiety is a PEG moiety having an oxocarboxylate residue. Any suitable oxocarboxylic acid derivative of PEG may be used. In a particular embodiment of the invention, the oxocarboxylate residue used is a pyruvoyl group.

In another embodiment of the invention, the PEG moiety having an aldehyde or ketone moiety is a PEG moiety having an aromatic ketone or aromatic aldehyde moiety, for example a benzaldehyde derivative of PEG.

In another embodiment of the invention, the PEG moiety having an aldehyde or ketone moiety is a PEG moiety having a trifluormethyl ketone moiety.

Any suitable interferon molecule may be used in the present invention. The terminal hydrazide moiety may be generated using any known technique in the art. As described in the Examples, the inventors were able to generate such C-terminal hydrazide moieties on interferon molecules by hydrazine induced cleavage of interferon molecules genetically fused N-terminally to an intein domain. Accordingly, in one embodiment of the first aspect of the present invention, the interferon molecule having a C terminal hydrazide moiety of step (b) is produced by reaction of hydrazine with a precursor molecule, said precursor molecule comprising a precursor interferon molecule fused N-terminally to an intein domain.

As described in the Examples, and to the inventors' surprise, on generating the interferon hydrazide using this method, it was found that the yield of cleaved interferon were significantly improved when the reaction was performed in the presence of a chelator, EDTA.

Accordingly, in one embodiment of the invention, in which the interferon molecule is produced by cleavage of a precursor interferon molecule fused to an intein domain, said precursor molecule is reacted with hydrazine in the presence of at least 10 μM, for example at least 0.1 mM, such as at least 0.2 mM, at least 0.5 mM, or at least 0.75 mM of a chelator. In such embodiments, any suitable chelator may be used. Chelators which may be used include DTPA, EDTA, or EGTA. In one such embodiment, the chelator is EDTA.

Moreover, as described in the Examples, the inventors found that, to their particular surprise, C-terminal hydrazide derivatives of the interferons produced by hydrazine cleavage of the corresponding intein fusion protein were isolated in their folded form. This is in contrast to conventional methods for producing interferons (alpha and betas), which when expressed in E. coli form inclusion bodies and require solubilisation and refolding to generate the active protein for PEGylation. As described in the Examples herein, the PEGylation method resulted in the generation of folded protein without the need for any refolding steps, or additives to promote protein folding. Protein folding and disulfide connectivity does not appear to be affected by the hydrazine cleavage step. This results in direct isolation of the folded C-terminal hydrazide protein after hydrazine cleavage of the precursor fusion protein.

Expression as the intein fusion appears to aid protein solubility in some cases. Protein folding and disulfide connectivity is not affected by the subsequent hydrazine cleavage step.

Thus, in one embodiment of the invention, in which the interferon molecule having a C terminal hydrazide moiety of step (b) is produced by reaction of hydrazine with a precursor interferon molecule fused N-terminally to an intein domain, the C-terminal hydrazide interferon protein obtained by hydrazine cleavage of the precursor interferon molecule fused N-terminally to the intein domain is obtained as a folded protein without any need for a refolding step or refolding agent.

Accordingly in one embodiment of the invention, the method is performed in the absence of a refolding step or refolding agent.

Thus in one embodiment, the C-terminal hydrazide interferon molecule in step (b) is a folded interferon molecule and the labelled interferon molecule formed in step (c) is a folded interferon molecule.

Furthermore another surprising advantage obtained for the interferons described in the Examples as produced using the methods of the invention was the enhanced activity compared to non selectively PEGylated interferon molecules.

Accordingly, in one embodiment of the invention, the labelled interferon molecule has antiviral activity of greater than 20% of that of the corresponding non-PEGylated interferon molecule. In particular embodiments of the present invention, the PEGylated interferon has at least 30%, for example at least 40%, for example at least 50%, such as at least 60%, at least 70%, at least 80% or at least 90% of the activity of the corresponding non-PEGylated interferon molecule.

The antiviral activity of the interferon molecules may be assessed using any suitable assay method known in the art. In one embodiment, the antiviral activity is assessed using a cytopathic effect inhibition assay using cancer cells, e.g. A549 lung cancer cells and suitable virus, e.g. EMC. An example of such a test is described in Example 3.4.

In particular embodiments of the invention, at least one of the label and the interferon comprises one or more disulphide bonds. A particular advantage of the labelling method of the invention is that it may be performed in the absence of thiols. This enables efficient ligation of proteins/peptides comprising disulphide bonds as well as of proteins without such bonds. Other labelling methods often require the presence of thiols such as 2-mercaptoethanesulfonic acid (MESNA), benzylmercaptan, thio phenol, (4-carboxylmethyl)thiophenol (MPPA).

The inventors have found that the reaction of aldehyde or ketone moiety of the PEG moiety with the C terminal hydrazide of the interferon molecule to form the labelled interferon molecule is enhanced by the presence of an aniline molecule, such as aniline or paramethoxy aniline, with both the rate of reaction and yield increased.

Accordingly, in one embodiment of the invention, step (c) is performed in the presence of an aniline molecule, such as aniline or paramethoxy aniline. The aniline or paramethoxy aniline may be employed at a concentration in the range 1-500 mM, for example, 5-200 mM, such as 5-100 mM. For example, where aniline is used, the range may be 1 to 50 mM and, for example, where paramethoxy aniline is used, the range may be 20 to 500 mM.

The method of the first aspect of the invention may be used to label any interferon. In a particular embodiment of the present invention, the interferon molecule is IFNalpha2b. In another embodiment, the interferon molecule is IFNbeta1b.

According to a second aspect of the invention, there is provided a C-terminal PEGylated interferon molecule, wherein the PEG moiety is attached to the C terminus of the interferon molecule via a hydrazone bond. In another embodiment, the PEG moiety is attached to the C terminus of the interferon molecule via a reduced hydrazone bond i.e. a substituted hydrazine i.e. a bond having formula

—NH—NH—CHR—

where R is H or any substituted or unsubstituted alkyl group.

In one embodiment of the first or second aspect of the invention, the interferon molecule is an IFNalpha2b molecule. In another embodiment, the interferon molecule is IFNbeta1b molecule.

In a particular embodiment of the first or second aspect of the invention, the interferon molecule is an IFNalpha2b molecule having amino acid sequence shown as Sequence ID No: 1, or a fragment or derivative thereof having at least 60%, such as at least 70%, for example at least 80%, at least 90%, or at least 95% sequence homology with Sequence ID No: 1:

Sequence ID No: 1:
CDLPQTHSLGSRRTLMLLAQMRRISLFSCLKDRHDFGFPQEEFGNQFQK
AETIPVLHEMIQQIFNLFSTKDSSAAWDETLLDKFYTELYQQLNDLEAC
VIQGVGVTETPLMKEDSILAVRKYFQRITLYLKEKKYSPCAWEVVRAEI
MRSFSLSTNLQESLRSKEG.

In one embodiment, the interferon molecule consists of the IFNalpha2b molecule having the amino acid sequence shown as Sequence ID No: 1.

In another particular embodiment of the of the first or second aspect of the invention the interferon molecule is an IFNbeta1b molecule having the amino acid sequence shown as Sequence ID No: 2, or a fragment or derivative thereof having at least 60%, for example at least 70%, at least 80%, or at least 90%, for example at least 95% sequence homology with Sequence ID No: 2:

Sequence ID No: 2
SYNLLGFLQRSSNFQSQKLLWQLNGRLEYCLKDRMNFDIPEEIKQLQQF
QKEDAALTIYEMLQNIFAIFRQDSSSTGWNETIVENLLANVYHQINHLK
TVLEEKLEKEDFTRGKLMSSLHLKRYYGRILHYLKAKEYSHCAWTIVRV
EILRNFYFINRLTGYLRNG.

In one embodiment, the interferon molecule consists of the IFNbeta 1b molecule having the amino acid sequence shown as Sequence ID No: 2.

In a particular embodiment of the invention the PEG moiety is a linear PEG moiety of approximately 10 kDa mass.

In one particular embodiment of the invention, the C-terminal PEGylated interferon molecule has formula

[Sequence ID No: 1]
-NH-N=CR-[PEG],

wherein R is —CH₃ and PEG is a linear PEG molecule of approximately 10 kDa mass.

In another particular embodiment of the invention, the C-terminal PEGylated interferon molecule has formula

[Sequence ID No: 2]
-NH-N=CR-[PEG],

wherein R is —CH₃ and PEG is a linear PEG molecule of approximately 10 kDa mass.

According to a third aspect of the invention, there is provided a PEGylated interferon molecule according to the second aspect of the invention or a PEGylated interferon produced according to the method of the first aspect of the invention for use in medicine.

A fourth aspect of the invention provides a method of treating a medical condition for which interferon treatment may be useful, in a patient in need thereof comprising administering a PEGylated interferon according to the second aspect of the invention or a PEGylated interferon produced according to the method of the first aspect of the invention. Such medical conditions include cancers, hepatitis C, multiple sclerosis, autoimmune disorders, and viral infections, for example influenza.

A fifth aspect of the invention provides a PEGylated interferon according to the second aspect of the invention or a PEGylated interferon produced according to the method of the first aspect of the invention for use in the treatment of cancer, hepatitis C, multiple sclerosis, an autoimmune disorder, or a viral condition.

A sixth aspect of the invention provides use of a PEGylated interferon according to the second aspect of the invention or a PEGylated interferon produced according to the method of the first aspect of the invention in the preparation of a medicament for the treatment of cancer, hepatitis C, multiple sclerosis, autoimmune disorders, or a viral condition.

A seventh aspect of the invention provides a pharmaceutical composition comprising a PEGylated interferon according to the second aspect of the invention or a PEGylated interferon produced according to the method of the first aspect of the invention.

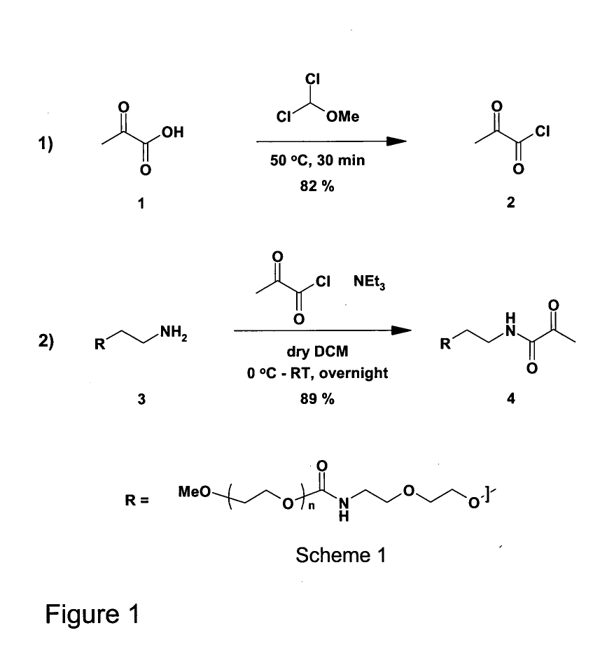

An eighth aspect of the invention provides a method of producing a labelled interferon molecule substantially as hereinbefore described with reference to any one of FIGS. 1 to 10.

A ninth aspect of the invention provides a C-terminal PEGylated interferon molecule substantially as hereinbefore described with reference to any one of FIGS. 1 to 10.

Preferred features of each aspect of the invention are as for each of the other aspects mutatis mutandis.

### DETAILED DESCRIPTION

Unless the context demands otherwise, the terms peptide, oligopeptide, polypeptide and protein are used interchangeably.

A method of site-specific C terminal PEGylation of interferons is provided which enables the generation of PEGylated interferons with considerable advantages over known PEGylated interferons. This was facilitated by generating a PEG moiety having an aldehyde or ketone moiety, such as an oxocarboxylic acid derivative of PEG, and its reaction with C terminal hydrazide interferon, which may optionally be produced through hydrazine cleavage of the corresponding intein fusion protein. This generates the site-specifically C terminal PEGylated protein, in which the PEG functionality is directly attached to the C terminus of the protein through a hydrazone bond.

### PEG

Any suitable polyethylene glycol may be used in the preset invention. In the context of the present invention, the term polyethylene glycol (PEG) is used synonymously with polyoxyethylene (POE). In the context of the present invention, the term polyethylene glycol (PEG) is used synonymously with polyoxyethylene (POE) and the PEG/POE may be of any suitable size.

In a particular embodiment of the invention, the PEG molecule has a mass in the range 1-60 KDa, such as 2-40 KDa, such as 2-20 kDa, for example in the range 5-18 kDa, such as 8-15 kDa, such as 19-12 KDa, such as approximately 10 kDa. In particular embodiment, the PEG molecule is a linear PEG molecule of approximately 10 kDa. The molecular weight may be ascertained using any suitable conventional technique, for example by gel filtration column chromatography with suitable weight markers, MALDI-TOF mass spectrometry etc.

The PEG may be, for example, linear, branched, star or comb PEG. Different forms of PEG are also available dependent on the initiator used for the polymerization process, as is well known to the skilled person.

The PEG molecule for use in the invention may be functionalised with any suitable aldehyde or ketone moiety.

In one embodiment, the aldehyde or ketone moiety is an α-diketone or an α-keto-aldehyde group.

In one embodiment, the PEG moiety having an aldehyde or ketone moiety has Formula II:

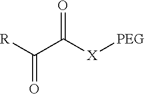

where X is a linker which may or may not be present, and R is a proton, H or any other functionality. In one embodiment R is a substituted or unsubstituted alkyl group. X, where present may be any suitable linker. In one embodiment X is NH. In another embodiment X is O. In another embodiment X is (CH₂)ₙ, where n is 0, 1, 2, 3, 4 or any whole number, for example a whole number in the range 5-100, for example in the range 5-50 or 5-10.

In further embodiments, the PEG moiety having an aldehyde or ketone moiety is a PEG moiety having an aromatic ketone or aromatic aldehyde moiety for example a benzaldehyde derivative of PEG. Such a PEG moiety is shown schematically as Formula III:

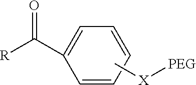

wherein R is a proton, H or another functionality; X, which may or may not be present, is defined as for Formula II, and PEG is attached at any position to the ring. The other positions of the ring may be substituted or unsubstituted. In one embodiment R is a substituted or unsubstituted alkyl group.

In another embodiment of the invention, the PEG moiety having an aldehyde or ketone moiety is a PEG moiety having a trifluoromethyl ketone moiety. Such a PEG moiety is shown schematically as Formula IV:

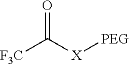

wherein X is a linker which may or may not be present. In one embodiment, X is as defined for Formula II.

In one embodiment, the PEG moiety having an aldehyde or ketone moiety is a PEG moiety having an oxocarboxylate residue. In one such embodiment, the PEG moiety having the oxocarboxylate residue has Formula V:

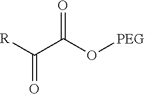

where R is a proton, H or another functionality. In one embodiment, R is a substituted or unsubstituted alkyl group.

Any suitable oxocarboxylate residue may be used, for example a pyruvoyl, gluoxyloyl (glyoxylyl), acetoacetyl, mesoxalyl, mesoxalo, oxalacetyl, or oxalaceto residue. In a particular embodiment of the present invention the PEG moiety having an oxocarboxylate residue is a pyruvoyl PEG. In another embodiment, the PEG moiety having an oxocarboxylate residue is a gluoxyloyl (glyoxylyl) PEG.

In one embodiment, the PEG moiety having an aldehyde or ketone moiety is considered to encompass a PEG moiety having a maleimide moiety. In another embodiment, the PEG moiety having an aldehyde or ketone moiety is considered not to encompass a PEG moiety having a maleimide moiety.

### Interferon Molecules

Interferon molecules of and for use in the present invention may be natural, recombinant or synthetic and may be of any interferon type, for example Type I Interferons such as IFN alpha, beta, lambda, omega, tau, kappa, epsilon, and zeta, Type II interferons such as IFN gamma, and Type III interferons such as IL-29, IL-28A and IL28B. In a particular embodiment of the present invention, the interferon molecule is IFNalpha2b. In another embodiment, the interferon molecule is IFNbeta1b. Encompassed by interferon molecules are fragments and derivatives of full length interferon molecules. Derivatives include analogues having at least 60%, for example at least 70%, 80% or 90% sequence homology with a corresponding sequence of natural interferon or fragment thereof. Such derivatives and fragments may optionally be coupled to additional peptidyl or non-peptidyl moieties. Preferably such fragments and derivatives retain therapeutic activity of an interferon, for example antiviral activity as described herein. In a particular embodiment of the present invention, the interferon molecule is IFNalpha2. In another embodiment, the interferon molecule is IFNbeta.

The interferon molecules of and for use in the invention may optionally have one or more additional amino acid residues at the C-terminal. In one embodiment, the interferon molecule is an interferon molecule with a Glycine addition at the C-terminal. In one embodiment, the interferon molecule is an IFNalpha2b molecule having amino acid sequence shown as Sequence ID No: 1, or a fragment or derivative thereof having at least 60%, at least 70%, at least 80%, at least 90%, for example at least 95% sequence homology with Sequence ID No: 1. In another particular embodiment, the interferon molecule is an IFNbeta 1b molecule having the amino acid sequence shown as Sequence ID No: 2, or a fragment or derivative thereof having at least 60%, at least 70%, at least 80%, at least 90%, for example at least 95% sequence homology with Sequence ID No: 2.

The interferon molecules of and for use in the invention may optionally have one or more additional amino acid residues at the N-terminal or both N- and C-terminal.

Hydrazide containing derivatives of synthetic oligopeptides may be readily produced using known methods, for example, solid phase synthesis techniques.

Carboxylic acid functionalities may be activated using carbodiimides and then reacted with hydrazine.

As described above, the present inventors have also found that interferons fused N-terminal to an intein domain can be cleaved from the intein by hydrazine treatment in a selective manner to liberate the desired interferon as its corresponding hydrazide derivative which can subsequently be used for reaction with aldehyde or ketone functional group of the PEG molecule, e.g. pyruvoyl PEG molecule to generate the PEGylated interferons according to the invention.

Such a method is based on the manipulation of a naturally occurring biological phenomenon known as protein splicing (Paulus H. Annu Rev Biochem 2000, 69, 447-496). Protein splicing is a post-translational process in which a precursor protein undergoes a series of intramolecular rearrangements which result in precise removal of an internal region, referred to as an intein, and ligation of the two flanking sequences, termed exteins. While there are generally no sequence requirements in either of the exteins, inteins are characterised by several conserved sequence motifs and well over a hundred members of this protein domain family have now been identified.

The first step in protein splicing involves an N→S (or N→O) acyl shift in which the N-extein unit is transferred to the sidechain SH or OH group of a conserved Cys/Ser/Thr residue, always located at the immediate N-terminus of the intein. Insights into this mechanism have led to the design of a number of mutant inteins which can only promote the first step of protein splicing (Chong et al Gene. 1997, 192, 271-281, (Noren et al., Angew. Chem. Int. Ed. Engl., 2000, 39, 450-466). Proteins expressed as in frame N-terminal fusions to one of these engineered inteins can be cleaved by thiols via an intermolecular transthioesterification reaction, to generate the recombinant protein C-terminal thioester derivative (Chong et al Gene. 1997, 192, 271-281, (Noren et al., Angew. Chem. Int. Ed. Engl., 2000, 39, 450-466)(New England Biolabs Impact System WO 00/18881, WO 0047751). Peptide sequences containing an N-terminal cysteine residue can then be specifically ligated to the C-termini of such recombinant C-terminal thioester proteins (Muir et al Proc. Natl. Acad. Sci. USA., 1998, 95, 6705-6710, Evans Jr et al. Prot. Sci., 1998, 7, 2256-2264), in a procedure termed expressed protein ligation (EPL) or intein-mediated protein ligation (IPL). One approach for the labelling of recombinant proteins is through the production of recombinant C terminal hydrazide proteins, by hydrazine cleavage of the corresponding intein fusion protein, and subsequent labelling through hydrazone bond forming reactions as described in WO2005/014620 A1. Briefly, the desired protein is expressed as an N terminal fusion of an engineered intein domain. Subsequent N to S acyl shift at the protein-intein union results in a thioester linked intermediate that can be chemically cleaved with hydrazine to give the desired protein C terminal hydrazide.

### Pharmaceutical Compositions

The PEGylated interferons may be administered as a pharmaceutical composition. Pharmaceutical compositions according to the present invention, and for use in accordance with the present invention, may comprise, in addition to active ingredients, a pharmaceutically acceptable excipient, a carrier, buffer stabiliser or other materials well known to those skilled in the art (see, for example, Remington: the Science and Practice of Pharmacy, 21^{st} edition, Gennaro A R, et al, eds., Lippincott Williams & Wilkins, 2005). Such materials may include buffers such as acetate, Iris, phosphate, citrate, and other organic acids; antioxidants; preservatives; proteins, such as serum albumin, gelatin, or immunoglobulins; hydrophilic polymers such aspolyvinylpyrrolidone; amino acids such as glycine, glutamine, asparagine, histidine, arginine, or lysine; carbohydrates; chelating agents; tonicifiers; and surfactants.

The pharmaceutical compositions may also contain one or more further active compound selected as necessary for the particular indication being treated, preferably with complementary activities that do not adversely affect the activity of the binding member, nucleic acid or composition of the invention. For example, in the treatment of cancer, in addition to the interferon, the composition may comprise a chemotherapeutic agent.

The active ingredients (e.g. interferon) may be administered via any suitable route and via any suitable means, for example microspheres, microcapsules, liposomes, other microparticulate delivery systems. For example, active ingredients may be entrapped within microcapsules which may be prepared, for example, by coacervation techniques or by interfacial polymerization, for example, hydroxymethylcellulose or gelatin microcapsules and poly-(methylmethacylate) microcapsules, respectively, in colloidal drug delivery systems (for example, liposomes, albumin microspheres, microemulsions, nano-particles and nanocapsules) or in macroemulsions. For further details, see Remington: the Science and Practice of Pharmacy, 21^{st} edition, Gennaro A R, et al, eds., Lippincott Williams & Wilkins, 2005.

Sustained-release preparations may be used for delivery of active agents. Suitable examples of sustained-release preparations include semi-permeable matrices of solid hydrophobic polymers containing the antibody, which matrices are in the form of shaped articles, e.g. films, suppositories or microcapsules. Examples of sustained-release matrices include polyesters, hydrogels (for example, poly(2-hydroxyethyl-methacrylate), or poly (vinylalcohol)), polylactides (U.S. Pat. No. 3,773,919), copolymers of L-glutamic acid and ethyl L-glutamate, non-degradable ethylene-vinyl acetate, degradable lactic acid-glycolic acid copolymers, and poly-D-(−)-3-hydroxybutyric acid.

Any suitable route of administration may be used to deliver the PEGylated interferons of the invention. In one embodiment, the interferons are delivered intramuscularly.

The active agent, product or composition may be administered in a localised manner to a tumour site or other desired site or may be delivered in a manner in which it targets tumour or other cells. Targeting therapies may be used to deliver the active agents more specifically to certain types of cell, by the use of targeting systems such as antibody or cell specific ligands. Targeting may be desirable for a variety of reasons, for example if the agent is unacceptably toxic, or if it would otherwise require too high a dosage, or if it would not otherwise be able to enter the target cells.

The active agents or compositions of the invention are preferably administered to an individual in a “therapeutically effective amount”, this being sufficient to show benefit to the individual. The actual dosage regimen will depend on a number of factors including the condition being treated, its severity, the patient being treated, the agent being used, and will be at the discretion of the physician. The optimal dose can be determined by physicians based on a number of parameters including, for example, age, sex, weight, severity of the condition being treated, the active ingredient being administered and the route of administration.

### Treatment

Treatment” includes any regime that can benefit a human or non-human animal. The treatment may be in respect of an existing condition or may be prophylactic (preventative treatment). Treatment may include curative, alleviation or prophylactic effects.

The PEGylated interferons of the invention may be used in the treatment of any condition for which interferon based treatment is useful. These may include neoplastic cancer, hepatitis, multiple sclerosis, autoimmune disorders, or viral conditions.

In one embodiment, the invention may be used in the treatment of cancer. “Treatment of cancer” includes treatment of conditions caused by cancerous growth and/or vascularisation and includes the treatment of neoplastic growths or tumours. Examples of tumours that can be treated using the invention are, for instance, sarcomas, including osteogenic and soft tissue sarcomas, carcinomas, e.g., breast-, lung-, bladder-, thyroid-, prostate-, colon-, rectum-, pancreas-, stomach-, liver-, uterine-, prostate, cervical and ovarian carcinoma, non-small cell lung cancer, hepatocellular carcinoma, lymphomas, including Hodgkin and non-Hodgkin lymphomas, neuroblastoma, melanoma, myeloma, Wilms tumor, and leukemias, including acute lymphoblastic leukaemia and acute myeloblastic leukaemia, astrocytomas, gliomas and retinoblastomas.

The invention may be particularly useful in the treatment of existing cancer and in the prevention of the recurrence of cancer after initial treatment or surgery.

In another embodiment, the invention may be used in the treatment of a viral infection, for example hepatitis C infection, influenza etc.

In another embodiment, the invention may be used in the treatment of multiple sclerosis.

In another embodiment, the invention may be used in the treatment of an, autoimmune disorder, for example lupus erythematosus.

In another embodiment, the invention may be used in the treatment of dependent diabetes mellitus (IDDM).

The invention will now be described further in the following non-limiting examples with reference made to the accompanying drawings in which:

FIG. 1 illustrates a scheme for preparation of A 10 kDa PEG target compound containing an N-terminal pyruvoyl functionality;

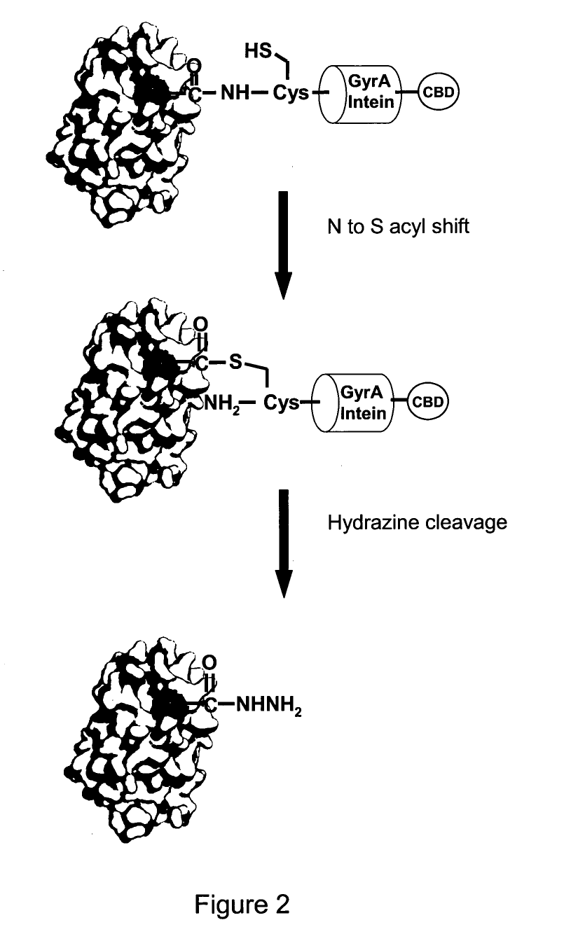

FIG. 2 illustrates schematically a method of generating a terminal hydrazide derivative of an interferon by hydrazine cleavage of the corresponding intein fusion protein;

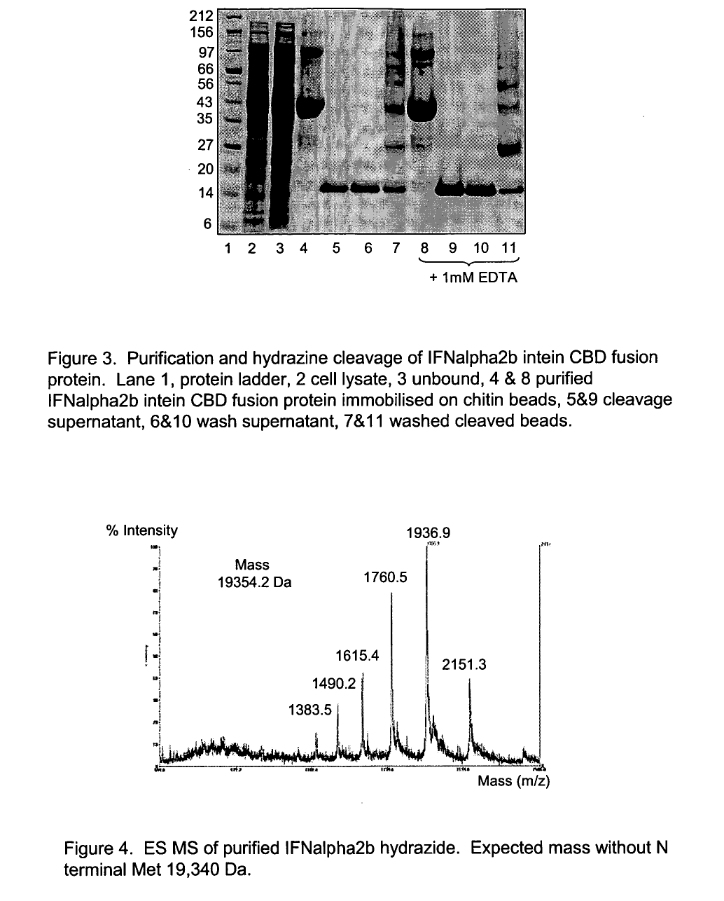

FIG. 3 illustrates a gel illustrating purification and hydrazine cleavage of IFNalpha2b intein CBD fusion protein;

FIG. 4 illustrates ES MS of purified IFNalpha2b hydrazide;

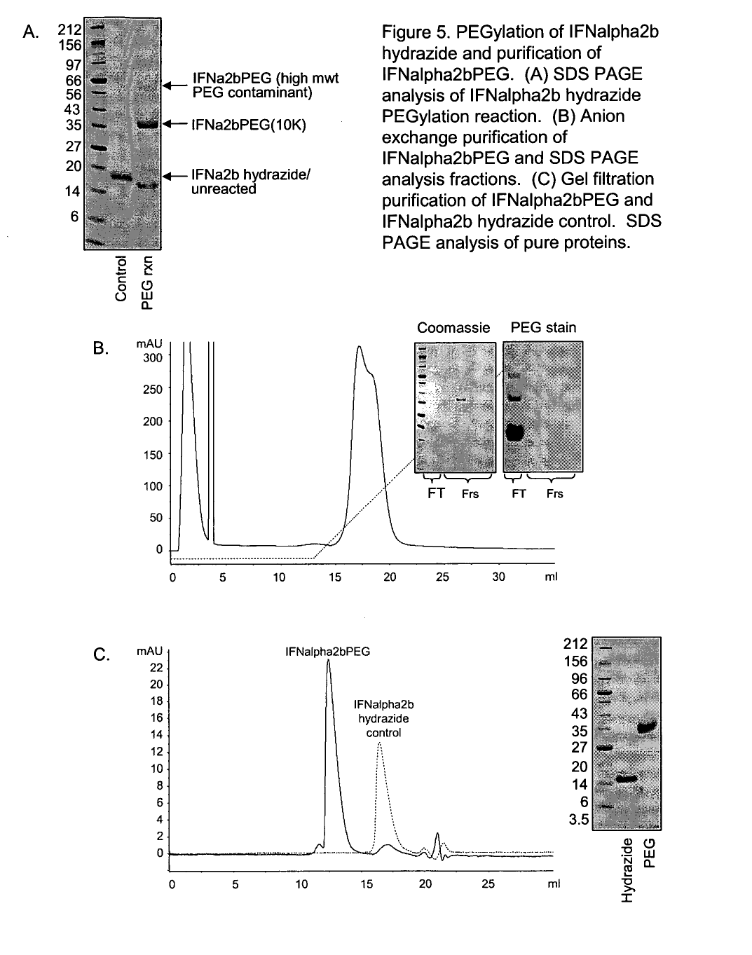

FIG. 5 illustrates SDS PAGE analysis of PEGylation of IFNalpha2b hydrazide& purification of IFNalpha2bPEG;

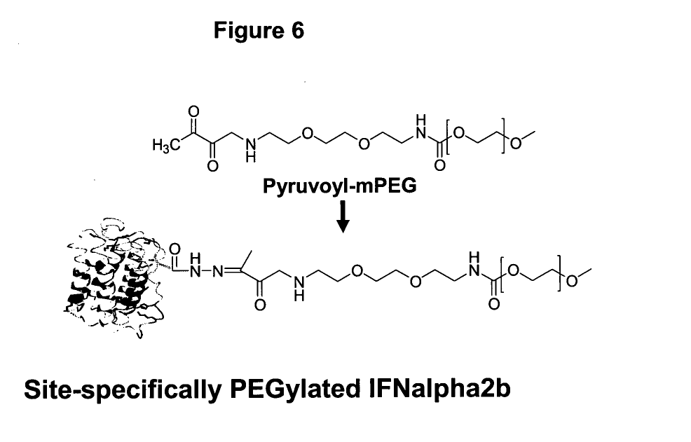

FIG. 6 illustrates schematically site-specifically PEGylated IFNa2b;

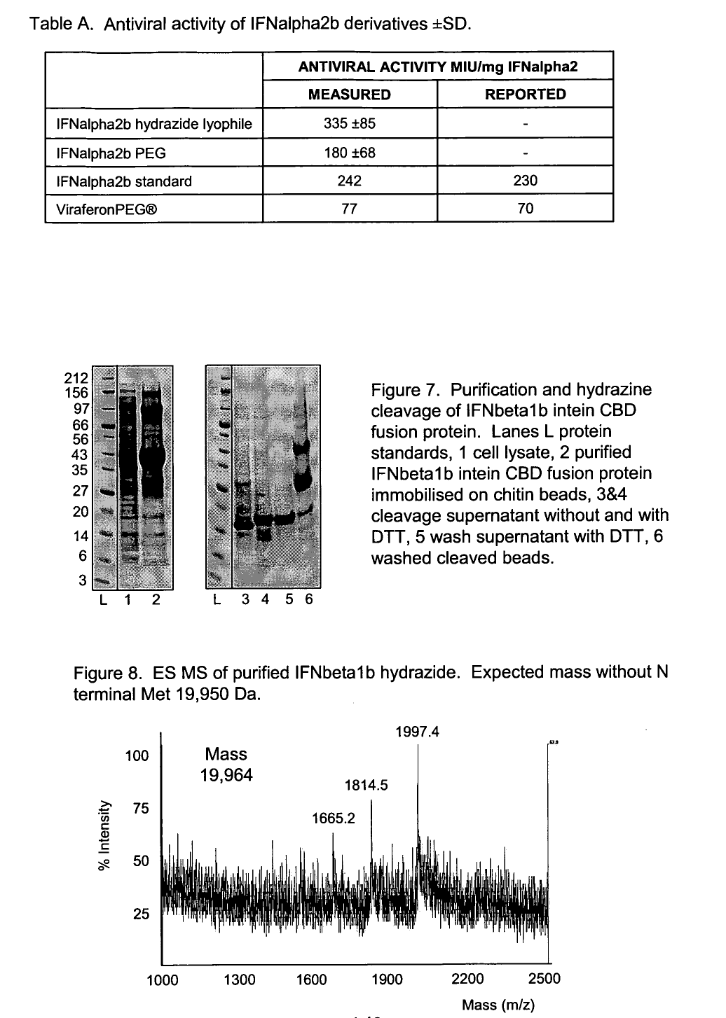

FIG. 7 illustrates gels used in the analysis of purification and hydrazine cleavage of IFNbeta1b intein CBD fusion protein and includes Table A;

FIG. 8 illustrates ES MS of purified IFNbeta1b hydrazide;

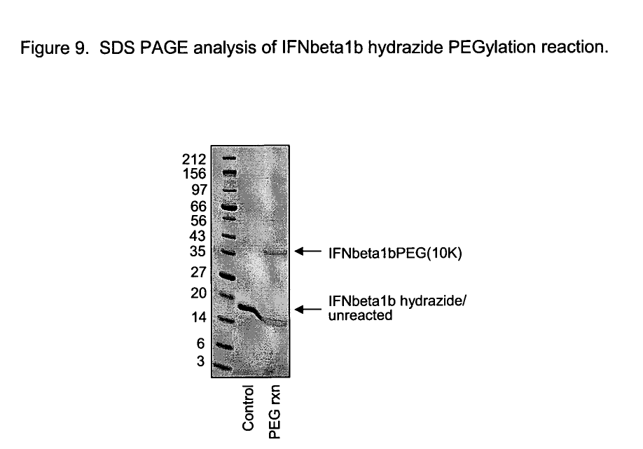

FIG. 9 illustrates SDS PAGE analysis of IFNbeta1b hydrazide PEGylation reaction;

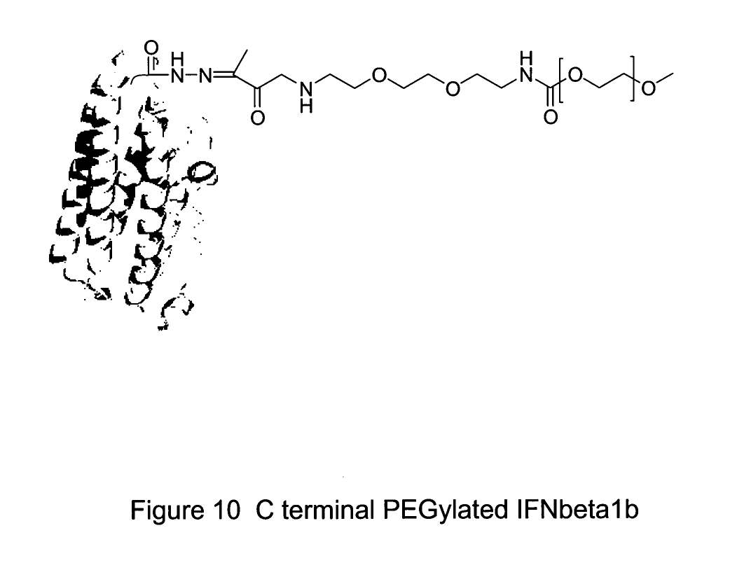

FIG. 10 illustrates schematically a C terminal PEGylated IFNbeta1b molecule; and

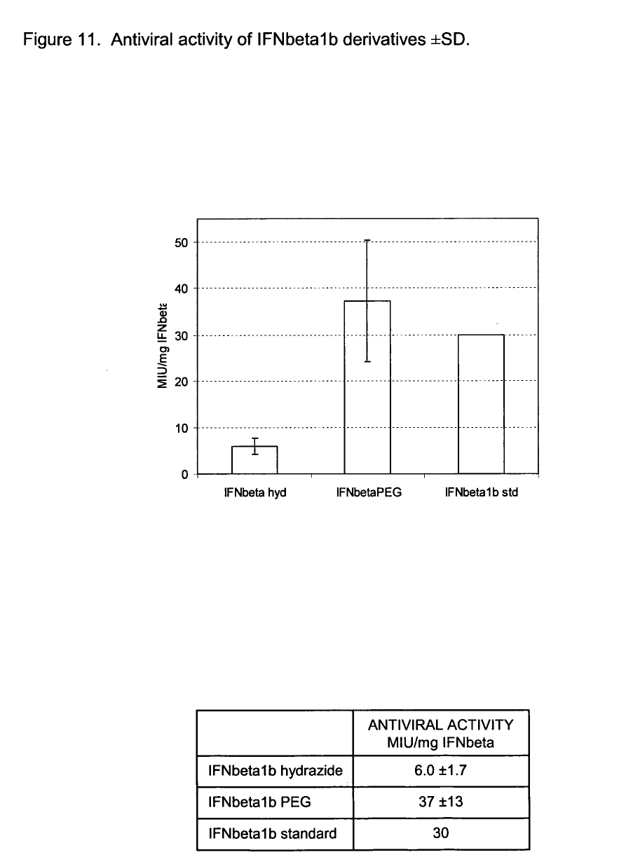

FIG. 11 illustrates a graph showing antiviral activity of IFNbeta1b derivatives ±SD.

### Example 1

### Generation of Pyruvoyl-PEG

A 10 kDa PEG target compound (4), containing an N-terminal pyruvoyl functionality, was prepared as shown in Scheme 1 of FIG. 1. This was achieved by overnight acylation of the commercially available PEG amine (3) with the preformed pyruvoyl chloride (2). The PEG amine was obtained from Nektar {MeO-PEG-NH2Nektar/2M2U0I01/PT03F24].

The acid chloride (2) was formed by treatment of pyruvic acid (1) with α,α-dichloromethyl methyl ether. Briefly, pyruvic acid (5 g) was charged to a 50 ml 3-necked RB flask, under nitrogen, equipped with a reflux condenser, a dropping funnel and connected to a dreschel bottle containing 2N NaOH (aq). α,α-dichloromethyl methylether (5.16 ml) was added dropwise, the reaction mixture was heated to 50° C. for 30 min, the methyl formate byproduct was removed by evaporation under reduced pressure, and the crude acid chloride was obtained as a yellow oil in 82% yield (4.96 g).

The crude acid chloride was obtained in 82% yield and was sufficiently pure (as determined by ¹H NMR) to be taken through the next step. The acid chloride is highly moisture sensitive. Exposure to moisture during a trial reaction resulted in partial decomposition of the product.

The target compound (4) was formed in 89% yield by overnight coupling between purified acid chloride (2) and PEG amine (3). Briefly, MeO-PEG-NH2 (500 mg) and anhydrous DCM (5 ml) were charged to a 50 ml RB flask under nitrogen. Triethyl amine (11 ml) was added and the reaction mixture was cooled to 0° C. Pyruvoyl chloride (10 mg) was added dropwise, keeping the temperature below 5° C. The reaction mixture was allowed to come back to room temperature overnight, the organic layer was washed with 2N HCl (2×10 ml) and then H₂O (10 ml), the organic layer was dried over Na₂SO₄, the solvent was removed under reduced pressure, the residue was slurried with Et20 to afford the pure product as a white solid in 82% yield (425 mg).

### Example 2

### Generation of Site Specifically C Terminal PEGylated IFNalpha2b Hydrazide

### Example 2.1

### Cloning, Expression and Purification of Soluble IFNalpha2B Hydrazide

IFNalpha2b cDNA (IMAGE clone 30915269) was purchased from Gene Service Ltd. The IFNalpha2b coding sequence was amplified by PCR using the following primers:

The forward primer was designed to include an NdeI site immediately prior to the 5′ IFNalpha2b sequence:

5′-GGTGGTCATATGTGTGATCTGCCTCAAACCC-3′

The reverse primer was designed to eliminate the STOP codon at the end of the IFNalpha2b coding sequence, replacing it with a glycine codon followed immediately with a SapI site:

5′-GGTGGTTGCTCTTCCGCACCCTTCCTTACTTCTTAAACTTTCTTG
C-3′

The resulting PCR product was cloned into the NdeI SapI sites of the pTXB1 vector (NEB). This pTXB1 IFNalpha2b GLY construct encodes a fusion protein whereby IFNalpha2b is linked via glycine to the N terminus of GyrA intein that is in turn fused to the N terminus of chitin binding domain (CBD). This was transformed into E. coli Rosetta gami B (DE3) pLysS cells (Novagen) and expression induced with 0.2 mM IPTG overnight at 18° C. Cells were pelleted by centrifugation and lysed in lysis buffer (20 mM sodium phosphate pH 7.4, 0.5 M NaCl, 0.5 mM EDTA, 15% glycerol, 0.1% Sarkosyl NL) with 1 mM AEBSF by sonication. The soluble fraction was mixed with chitin beads pre-equilibrated in lysis buffer, 4° C. for 1.5 hours. The beads were then washed extensively with lysis buffer followed by ligation buffer (200 mM sodium phosphate pH 7.4, 200 mM NaCl, 0.05% Zwittergent 3-14) to yield purified IFNalpha2b GyrA intein CBD fusion protein immobilised on chitin beads (FIG. 3 lane 4).

Overnight treatment of these beads with 1% hydrazine in ligation buffer generated IFNalpha2b hydrazide. The reaction is shown schematically in FIG. 2. Addition of 1 mM EDTA to the cleavage resulted in greater yields of cleaved material (FIG. 3 compare lanes 5 & 9). Without being limited to any one theory, it is possible that EDTA mops up trace metal ions that could potentially inhibit the activity of the intein. IFNalpha2b hydrazide was purified by RP HPLC on a Jupiter C5 column (Phenomenix) in water with 0.1% TFA and an acetonitrile 0.1% TFA gradient to give the pure protein lyophile. Expected mass IFNalpha2b without the N terminal met=19,340 Da; observed mass=19,336 Da; typical yield is ˜0.7 mg/L cell culture (FIG. 4). The sequence of the IFNalpha2b hydrazide made here is:

CDLPQTHSLGSRRTLMLLAQMRRISLFSCLKDRHDFGFPQEEFGNQFQK
AETIPVLHEMIQQIFNLFSTKDSSAAWDETLLDKFYTELYQQLNDLEAC
VIQGVGVTETPLMKEDSILAVRKYFQRITLYLKEKKYSPCAWEVVRAEI
MRSFSLSTNLQESLRSKEG-NHNH₂

Reaction with N-ethyl maleimide (NEM) was used to confirm that this IFNalpha2b hydrazide was correctly folded. NEM reacts with free cysteines resulting in an increase in mass of 125. IFNalpha2b has 4 cysteines and 2 disulfide bonds, and therefore the folded protein will not increase in mass on incubation with NEM. A few μg of pure IFNalpha2b hydrazide was dissolved in 20 μl water or 40% acetonitrile. 10 μl was removed for the control and 5 μl 1 mg/ml NEM was added to remaining and incubated at room temperature for at least 30 mins then analysed by ES MS analysis. IFNalpha2b did not react with NEM in contrast to the positive control (peptide sequence CERGDKGYVPSVF) that increased in mass by 125 Da. Accordingly, the results show that the folded C terminal hydrazide derivative of IFNalpha2b was directly produced after expression and hydrazine cleavage of the corresponding intein fusion protein.

### Example 2.2

### PEGvlation of IFNalpha2b Hydrazide

20 fold molar excess of pyruvoyl-PEG was dissolved in 100 μl 40% acetonitrile with 0.1% TFA and added to the IFNalpha2b lyophile. Reactions left at room temperature overnight (˜16 hours) and analysed on NuPAGE 4-12% Bis-Tris gels in MES running buffer under reducing conditions (FIG. 5A). No PEGylation product was observed when IFNalpha2b C terminal thioester was incubated with pyruvoyl PEG under the same conditions, consistent with site specific PEGylation via the C terminal hydrazide group only. The reaction is shown schematically in FIG. 6.

### Example 2.3

### Purification of PEGylated IFNalpha2b

Firstly, ion exchange was used to remove unreacted pyruvoyl-PEG. PEG is uncharged therefore it will not bind ion exchange columns, in contrast to proteins that are charged. The 100 μl IFNalpha2b PEG reaction was made up to 1 ml in buffer A (20 mM Tris pH7.3, 0.05% Zwittergent 3-14) and loaded onto a 1 ml HiTrap Q FF anion exchange column via AKTA purifier system (GE Healthcare). The column was washed with 5-10 CV buffer A to remove unbound, unreacted pyruvoyl-PEG and the bound protein eluted over a 0 to 1 M NaCl gradient (20 CV). Fractions were analysed on a NuPAGE 4-12% Bis-Tris gel in MES running buffer under reducing conditions (FIG. 5B). The gel was run in duplicate; one was stained with coomassie and the other was stained for PEG, based on the methods in the literature (Kurfurst, 1992; Lee et al., 2008) Briefly, the gel was rocked in 20 ml of 0.1 M perchloric acid for 15 mins, then transferred to 5 ml 5% wt/vol barium chloride solution and 2 ml 0.1 M iodine for 10 mins and destained in water. This clearly shows that the unreacted pyruvoyl-PEG did not bind the column as expected. The fractions containing the desired IFNalpha2bPEG were concentrated using VivaSpin2 3K MWCO centrifugal concentrators (Sartorius). This was run through a Superdex 200 10/300 GL column (GE Healthcare) in 10 mM sodium phosphate pH 7.4, 50 mM NaCl, 0.05% Zwittergent 3-14 to separate PEGylated IFNalpha2b from the unreacted IFNalpha2b hydrazide. The pooled fractions were analysed by SDS PAGE with coomassie staining (FIG. 5C).

### Example 2.4

### Antiviral Activity of IFNalpha2bPEG and the Hydrazide Control

The anti-viral activities of the purified IFNalpha2bPEG and IFNalpha2b hydrazide control, that had been through the same purification and handling steps as the PEGylated molecule, were determined using a cytopathic effect inhibition assay with human A549 lung carcinoma cells & EMC virus (run by PBL Interferon Source) (Table A). The activity of the IFNalpha2b hydrazide was higher than the IFNalpha2b standard, perhaps due to initial purification of the folded material rather than refolding from inclusion bodies as is the case with standard IFNalpha2b. Alternatively, it is possible that the presence of the hydrazide group at the C terminus may be advantageous, for example by decreasing susceptibility of the C terminus to exoproteases. Activity of the site specifically C terminal PEGylated IFNalpha2b (180±68 U/mg) is more than double that of the heterogeneous PEGylated preparation of ViraferonPEG (77 MIU/mg measured and 70 MIU/mg reported).

The activity of C terminal PEGylated IFNalpha2b is significantly higher than the activity of the heterogeneously PEGylated ViraferonPEG (77 MIU/mg measured and 70 MIU/mg reported).

### Example 3

### Generation of Site Specifically C Terminal PEGylated IFNbeta1b Hydrazide

### Example 3.1

### Cloning, Expression and Purification of Soluble IFNbeta1B Hydrazide

DNA encoding the IFNbeta1b protein sequence with an additional C terminal glycine

SYNLLGFLQRSSNFQSQKLLWQLNGRLEYCLKDRMNFDIPEEIKQLQQF
QKEDAALTIYEMLQNIFAIFRQDSSSTGWNETIVENLLANVYHQINHLK
TVLEEKLEKEDFTRGKLMSSLHLKRYYGRILHYLKAKEYSHCAWTIVRV
EILRNFYFINRLTGYLRNG

was optimised for expression in E. coli and synthesised by GeneArt with the following flanking DNA sequence containing a 5′ NdeI site and a 3′ SapI site:

5′-GGT GGT CAT . . . [IFNbeta1b sequence] . . . TGC GGA AGA GCA
ACC ACC-3′

Digestion of the supplied DNA with NdeI and SapI resulted in an IFNbeta1b fragment that could be directly ligated into similarly digested pTXB1 vector. This pTXB1 IFNbeta1b GLY construct encodes a fusion protein whereby IFNbeta1b is linked via glycine to the N terminus of GyrA intein that is in turn fused to the N terminus of chitin binding domain (CBD). This was transformed into E. coli Origami (DE3) cells (Novagen) and expression induced with 0.2 mM IPTG overnight at 18° C. Cells were pelleted by centrifugation and lysed in lysis buffer (20 mM sodium phosphate pH 7.4, 0.5 M NaCl, 0.5 mM EDTA, 15% glycerol, 0.1° A Sarkosyl NL) with 1 mM AEBSF by sonication. The soluble fraction was mixed with chitin beads pre-equilibrated in lysis buffer, 4° C. for 1.5 hours. The beads were then washed extensively with lysis buffer followed by ligation buffer (200 mM sodium phosphate pH 7.4, 200 mM NaCl, 0.05% Zwittergent 3-14) to yield purified IFNbeta1b GyrA intein CB fusion protein immobilised on chitin beads (FIG. 7 lane 2).

Overnight treatment of these beads with 1% hydrazine and 1 mM EDTA in ligation buffer generated IFNbeta1b hydrazide (FIG. 7 lane 3). IFNbeta1b hydrazide treated with DTT gave a slower running band on SDS PAGE analysis (FIG. 7 compare lanes 3 & 4), consistent with the disulfide bond being formed in the recovered species and becoming reduced on DTT treatment. The expected mass of IFNbeta1b without the N terminal met=19,950 Da; observed mass=19,964 Da (FIG. 8). IFNbeta1b hydrazide was purified on a Superdex 75 column (GE Healthcare) in 3 mM acetic acid pH 3.7 with 0.05% Zwittergent 3-14 to give the pure protein hydrazide. NEM reactions were performed as described in example 3.1 to probe for protein folding. There was no increase in the mass of IFNbeta1b on incubation with NEM in contrast to the positive control, indicating that the disulfide bond is intact and the protein is correctly folded. Aliquots were lyophilised with 50 ug mannitol ug IFNbeta1b hydrazide. These aliquots were redissolved in 10 mM sodium phosphate pH 7.4 to give final buffer composition 10 mM sodium phosphate pH 7.4, 50 mM NaCl, 0.05% Zwittergent 3-14, 13.7 mM mannitol) and used as the IFNbeta hydrazide control.

### Example 3.2

### PEGylation of IFNbeta1b

The concentration of IFNbeta1b in the Superdex 75 fractions above was estimated from the absorbance at 280 nm and added to 200 fold molar excess of pyruvoyl-PEG. The reaction was sat at 4° C. overnight then analysed on a NuPAGE 4-12% Bis-Tris gel in MES running buffer under reducing conditions and stained with coomassie (FIG. 9). The PEGylated IFNbeta1b is shown schematically in FIG. 10.

### Example 3.3

### Purification of PEGylated IFNbeta1b

Ion exchange was used as the first step, to remove unreacted pyruvoyl-PEG. The IFNbeta1b PEG reaction was diluted 5 fold in buffer A (25 mM sodium phosphate pH 7.4, 0.05% Zwittergent 3-14) and loaded onto a 1 ml HiTrap SP XL cation exchange column via AKTA purifier system (GE Healthcare). The column was washed with 5 CV buffer A to remove unbound, unreacted pyruvoyl-PEG and the bound protein eluted over a 0 to 0.5 M NaCl gradient (20 CV). Fractions analysed by Western blot using sheep polyclonal anti human IFNbeta primary antibody (PBL Interferon Source) and rabbit anti sheep HRP conjugated secondary antibody (Invitrogen). The fractions containing IFNbeta1bPEG were concentrated using VivaSpin2 3K MWCO centrifugal concentrators (Sartorius) and run through a Superdex 200 10/300 GL column (GE Healthcare) in PBS with 0.05% Zwittergent 3-14 to separate PEGylated IFNbeta2b from the unreacted IFNbeta2b hydrazide. The fractions were analysed by Western blot as above and pure fractions were concentrated, aliquotted and lyophilised with 50 ug mannitol/ug IFNbeta1b hydrazide.

### Example 3.4

### Antiviral Activity of IFNbeta1bPEG and IFNbeta1B Hydrazide

Anti-viral activities of the purified IFNbeta1b hydrazide and IFNbeta1bPEG were determined using a cytopathic effect inhibition assay with human A549 lung carcinoma cells & EMC virus (PBL Interferon Source) (FIG. 11). The activity of IFNbeta1b hydrazide was lower than that of the IFNbeta1b standard, probably due to instability of the protein and the lack of stabilizing ingredients in the formulation. Site specifically C terminal PEGylated IFNbeta1b showed greater activity than the hydrazide, probably reflecting the increased protein stability brought by the PEG, and this activity was in line with that of the unPEGylated IFNbeta1b standard. (The activity of C terminal PEGylated IFNbeta1b (37±13 MIU/mg) is comparable to the non-PEGylated IFNbeta1b standard (30 MIU/mg)). There is currently no approved PEGylated version of IFNbeta1b.

All documents referred to in this specification are herein incorporated by reference. Various modifications and variations to the described embodiments of the inventions will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes of carrying out the invention which are obvious to those skilled in the art are intended to be covered by the present invention.

### REFERENCE LIST

Basu, A., Yang, K., Wang, M., Liu, S., Chintala, R., Palm, T., Zhao, H., Peng, P., Wu, D., Zhang, Z., Hua, J., Hsieh, M. C., Zhou, J., Petti, G., Li, X., Janjua, A., Mendez, M., Liu, J., Longley, C., Zhang, Z., Mehlig, M., Borowski, V., Viswanathan, M., and Filpula, D. (2006). Structure-function engineering of interferon-beta-1 b for improving stability, solubility, potency, immunogenicity, and pharmacokinetic properties by site-selective mono-PEGylation. Bioconjug. Chem. 17, 618-630.
Brocchini, S., Godwin, A., Balan, S., Choi, J. W., Zloh, M., and Shaunak, S. (2008). Disulfide bridge based PEGylation of proteins. Adv. Drug Deliv. Rev. 60, 3-12.
DeFrees, S., Wang, Z. G., Xing, R., Scott, A. E., Wang, J., Zopf, D., Gouty, D. L., Sjoberg, E. R., Panneerselvam, K., Brinkman-Van der Linden E C, Bayer, R. J., Tarp, M. A., and Clausen, H. (2006). GlycoPEGylation of recombinant therapeutic proteins produced in Escherichia coli. Glycobiology 16, 833-843.
Dhalluin, C., Ross, A., Leuthold, L. A., Foser, S., Gsell, B., Muller, F., and Senn, H. (2005). Structural and biophysical characterization of the 40 kDa PEG-interferon-alpha2a and its individual positional isomers. Bioconjug. Chem. 16, 504-517.
Dirksen, A., Hackeng, T. M., and Dawson, P. E. (2006). Nucleophilic catalysis of oxime ligation. Angew. Chem. Int Ed Engl. 45, 7581-7584.
Doores, K. J., Gamblin, D. P., and Davis, B. G. (2006). Exploring and exploiting the therapeutic potential of glycoconjugates. Chemistry 12, 656-665.
Ferrantini, M., Capone, I., and Belardelli, F. (2007). Interferon-alpha and cancer: mechanisms of action and new perspectives of clinical use. Biochimie 89, 884-893.
Fine, H. A., Wen, P. Y., Robertson, M., O'Neill, A., Kowal, J., Loeffler, J. S., and Black, P. M. (1997). A phase I trial of a new recombinant human beta-interferon (BG9015) for the treatment of patients with recurrent gliomas. Clin. Cancer Res 3, 381-387.
Foser, S., Schacher, A., Weyer, K. A., Brugger, D., Dietel, E., Marti, S., and Schreitmuller, T. (2003). Isolation, structural characterization, and antiviral activity of positional isomers of monopegylated interferon alpha-2a (PEGASYS). Protein Expr. Purif. 30, 78-87.
Fukutomi, T., Nakamuta, M., Fukutomi, M., Iwao, M., Watanabe, H., Hiroshige, K., Tanabe, Y., and Nawata, H. (2001). Decline of hepatitis C virus load in serum during the first 24 h after administration of interferon-beta as a predictor of the efficacy of therapy. J. Hepatol. 34, 100-107.
Kinstler, O., Molineux, G., Treuheit, M., Ladd, D., and Gegg, C. (2002). Mono-N-terminal poly(ethylene glycol)-protein conjugates. Adv. Drug Deliv. Rev. 54, 477-485.
Kurfurst, M. M. (1992). Detection and molecular weight determination of polyethylene glycol-modified hirudin by staining after sodium dodecyl sulfate-polyacrylamide gel electrophoresis. Anal. Biochem. 200, 244-248.
Lee, D. L., Sharif, I., Kodihalli, S., Stewart, D. I., and Tsvetnitsky, V. (2008). Preparation and characterization of monopegylated human granulocyte-macrophage colony-stimulating factor. J Interferon Cytokine Res 28, 101-112.
Marsac, Y., Cramer, J., Olschewski, D., Alexandrov, K., and Becker, C. F. (2006). Site-specific attachment of polyethylene glycol-like oligomers to proteins and peptides. Bioconjug. Chem. 17, 1492-1498.
Piehler, J., Roisman, L. C., and Schreiber, G. (2000). New structural and functional aspects of the type I interferon-receptor interaction revealed by comprehensive mutational analysis of the binding interface. J. Biol. Chem. 275, 40425-40433.
Roberts, M. J., Bentley, M. D., and Harris, J. M. (2002). Chemistry for peptide and protein PEGylation. Adv. Drug Deliv. Rev. 54, 459-476.
Subramanian, G. M., Fiscella, M., Lamouse-Smith, A., Zeuzem, S., and McHutchison, J. G. (2007). Albinterferon alpha-2b: a genetic fusion protein for the treatment of chronic hepatitis C. Nat. Biotechnol. 25, 1411-1419.
Veronese, F. M. and Mero, A. (2008). The impact of PEGylation on biological therapies. BioDrugs. 22, 315-329.
Wang, Y. S., Youngster, S., Grace, M., Bausch, J., Bordens, R., and Wyss, D. F. (2002). Structural and biological characterization of pegylated recombinant interferon alpha-2b and its therapeutic implications. Adv. Drug Deliv. Rev. 54, 547-570.
Xie, J. and Schultz, P. G. (2006). A chemical toolkit for proteins—an expanded genetic code. Nat. Rev. Mol. Cell. Biol. 7, 775-782.
Zhang, X., Li, F., Lu, X. W., and Liu, C. F. (2009). Protein C-terminal modification through thioacid/azide amidation. Bioconjug. Chem. 20, 197-200.
Zhao, H., Yang, K., Martinez, A., Basu, A., Chintala, R., Liu, H. C., Janjua, A., Wang, M., and Filpula, D. (2006). Linear and branched bicin linkers for releasable PEGylation of macromolecules: controlled release in vivo and in vitro from mono- and multi-PEGylated proteins. Bioconjug. Chem. 17, 341-351.

1. A method of site specific labelling of an interferon molecule, wherein said method comprises the steps:
a) providing a label molecule, the label molecule comprising a PEG moiety having an aldehyde or ketone moiety;
b) providing an interferon molecule, the interferon molecule having a C terminal hydrazide moiety;
c) allowing the aldehyde or ketone moiety of the PEG moiety to react with the C terminal hydrazide of the interferon molecule to form a labelled interferon molecule, which comprises a PEG moiety attached to the C terminus of the interferon molecule via a hydrazone bond.

2. The method according to claim 1, wherein said PEG moiety having an aldehyde or ketone moiety is a PEG moiety having an α-diketone or an α-keto-aldehyde group.

3. The method according to claim 1, wherein said PEG moiety having an aldehyde or ketone moiety is a PEG moiety having an oxocarboxylate residue.

4. The method according to claim 1, wherein said aldehyde or ketone moiety is an aromatic ketone or aromatic aldehyde moiety.

5. The method according to claim 3, wherein said oxocarboxylate residue is a pyruvoyl group.

6. The method according to claim 1, wherein the interferon molecule having a C terminal hydrazide moiety of step (b) is produced by reaction of hydrazine with a precursor molecule, said precursor molecule comprising a precursor interferon molecule fused N-terminally to an intein domain via a thioester moiety.

7. The method according to claim 6, wherein the interferon molecule having a C terminal hydrazide moiety of step (b) produced by reaction of hydrazine with a precursor molecule is produced directly as a folded protein without a refolding step or a refolding agent.

8. The method according to claim 6, wherein said precursor molecule is reacted with hydrazine in the presence of at least 0.1 mM of a chelator, for example EDTA.

9. The method according to claim 1, wherein the interferon molecule is an IFNalpha2b molecule having amino acid sequence shown as Sequence ID No: 1, or a fragment or derivative thereof having at least 60% sequence homology with Sequence ID No: 1.

10. The method according to claim 9, wherein the interferon molecule is IFNalpha2b.

11. The method according to claim 1, wherein the interferon molecule is an IFNbeta1b molecule having the amino acid sequence shown as Sequence ID No: 2, or a fragment or derivative thereof having at least 60% sequence homology with Sequence ID No: 2.

12. The method according to claim 11, wherein the interferon molecule is IFNbeta1b.

13. The method according to claim 1, wherein said labelled interferon molecule has antiviral activity of greater than 40% of that of the corresponding non-PEGylated interferon molecule.

14. A C-terminal PEGylated interferon molecule, wherein the PEG moiety is attached to the C terminus of the interferon molecule via a hydrazone bond, or a reduced hydrazone bond.

15. The C-terminal PEGylated interferon molecule according to claim 14, wherein the interferon molecule is an IFNalpha2b molecule having amino acid sequence shown as Sequence ID No: 1, or a fragment or derivative thereof having at least 60% sequence homology with Sequence ID No: 1.

16. The C-terminal PEGylated interferon molecule according to claim 15, wherein said interferon molecule is IFNalpha2b.

17. The C-terminal PEGylated interferon molecule according to claim 14, wherein said interferon molecule is an IFNbeta1b molecule having the amino acid sequence shown as Sequence ID No: 2, or a fragment or derivative thereof having at least 60% sequence homology with Sequence ID No: 2.

18. The C-terminal PEGylated interferon molecule according to claim 17, wherein said interferon molecule is IFNbeta1b.

19. The C-terminal PEGylated interferon molecule according to claim 14, wherein the PEG molecule is a linear PEG molecule of mass in the range 9 kDa to 12 kDa, such as approximately 10 kDa mass.

20. A method of treating a medical condition for which interferon treatment may be useful, in a patient in need thereof, comprising administering a PEGylated interferon produced according to the method of claim 1.

21. The method according to claim 20, wherein the medical condition is selected from the group consisting of cancer, IDDM, hepatitis C, multiple sclerosis, autoimmune disorder, and a viral condition.

22. A PEGylated interferon produced according to the method of claim 1 for use in medicine.

23. A PEGylated interferon produced according to the method of claim 1 for treatment of cancer, IDDM, hepatitis C, multiple sclerosis, an autoimmune disorder, or a viral condition.

24. Use of a PEGylated interferon produced according to the method of claim 1 in the preparation of a medicament for the treatment of cancer, IDDM, hepatitis C, multiple sclerosis, an autoimmune disorder, or a viral condition.

25. A pharmaceutical composition comprising a PEGylated interferon produced according to the method of claim 1.

26-27. (canceled)
